(19) United States
(12) Patent Application Publication
(10) Pub. No.: US 2009/0217390 A1
(43) Pub. Date: Aug. 27, 2009

(54) Non-human animal sarcoma model

(76) Inventors: Fernando Lecanda Cordero, Navarra (ES); Iranzu Gonzalez De La Tajada, Navarra (ES)

Correspondence Address: WENDEROTH, LIND & PONACK, L.L.P., 1030 15th Street, N.W.,, Suite 400 East, Washington, DC, 20005-1503, US

(21) Appl. No.: 11/919,487

(22) PCT Filed: Apr. 29, 2005
(86) PCT No.: PCT/ES2005/000234

§ 371 Date: Aug. 21, 2008

(51) Int. Cl. A01K 67/027; C12Q 1/68; C12N 5/00; C12Q 1/02

(52) U.S. Cl. 800/3; 800/21; 800/10; 435/6; 435/325; 435/29

(57) ABSTRACT

The invention disclosed in this application relates to a method designed to obtain a non-human animal model for sarcoma characterised in that it comprises: transfecting cells derived from a multipotent mesenchymal cell line with an expression vector that contains a polynucleotide which encodes a fusion protein selected from EWS-FLI1, EWS-ERG, EWS-ETV1, EWS-ETV4, EWS-FEV, EWS-ATF1, EWS-WT1, EWS-NR4A3 and EWS-DDIT3; and injecting said transfected cells in the animal that is the model subject. Similarly, this invention relates to the animal model itself and its applications, as well as the transfected cell line and the applications thereof.

### TECHNICAL FIELD OF THE INVENTION

This invention pertains to the field of non-human animal models; in this case, it is a model for the study of sarcomas.

### STATE OF THE ART PRIOR TO THE INVENTION

A sarcoma is a general type of infrequent cancer, in which cancer cells arise from, or resemble, the normal body cells that belong to “connective tissues”. Normal “connective tissues” include fatty tissue, muscle, blood vessels, deep skin tissues, nerves, bones and cartilage. Cancers of cells that resemble any of these normal tissues receive the name of sarcomas. In turn, sarcomas are sub-classified on the basis of the specific cell type that makes up the tumour. Some of the most common subtypes of sarcoma are mentioned below:
Liposarcomas are malignant tumours that develop from fatty tissue. They may develop in any part of the body, but most often they grow in the retroperitoneum.
Leiomyosarcomas are malignant tumours that develop from flat muscle. They may grow in any part of the body, but the uterus and the gastrointestinal tract are two relatively frequent locations for this type of tumour.
Rhabdomyosarcomas are malignant tumours that resemble developing skeletal muscle. These tumours mostly appear in the arms or legs, but they may also develop in the head or neck area, in addition to the urinary and reproductive organs.
Synovial sarcoma is a malignant tumour composed of cells that resemble the cells present in the joints (synovial cells, which line the joints). However, these tumours do not necessarily develop in a joint, and the name may be misleading, since the cells have a different origin. Synovial sarcomas develop in any part of the body and they frequently appear in young adults.
Ewing's sarcoma, also known as peripheral neuroectodermal tumour, has its origin in very primitive body cells.
Angiosarcoma is a malignant tumour that resembles blood or lymphatic vessels.
Fibrosarcoma is a cancer of fibroblast-type cells.
Malignant peripheral nerve sheath tumour (MPNST), also known as neurofibrosarcoma.
Gastrointestinal stromal tumour is a tumour of the connective cells that support the gastrointestinal tract.
Osteosarcoma is a bone cell tumour.
Chondrosarcoma is a tumour of the cells that form the cartilage.

Depending on the molecular alterations which cause said sarcomas, they may be classified as sarcomas arising from non-recurrent genetic aberrations and sarcomas characterised by the presence of a recurrent genetic alteration, such as a chromosomal translocation, as is the case in synovial sarcoma, myxoid sarcoma, clear cell sarcoma or Ewing's tumour. In general, these chromosomal translocations lead to chimeric transcription factors that deregulate the expression of their target genes. One of the most frequent genes that form a part of these chimeric proteins is the EWS gene.

Ewing's tumours represent the second most frequent solid tumour neoplasia in children. These tumours appear in the bone and in soft tissue, and arise from an unknown cell type. These tumours are characterised by the presence of specific chromosomal translocations that juxtapose the EWS gene at 22q12 to a gene belonging to the ETS family, the most common being FLI1 at 11q24. The EWS-FLI1 fusion protein is the most prevalent fusion, with an incidence of 95% of tumours, although EWS may also be translocated with other genes. This chimeric protein acts as an aberrant transcription factor with oncogenic properties.

At the genomic level, the breakpoints for the EWS gene extend along an 8-kb region, whilst in the case of FLI-1 they are dispersed along approximately 35 kb. This leads to multiple types of fusions that incorporate different exons for each gene. 12 types of EWS-FLI1 fusions have been described, although the most frequent ones are type 1, EWS exon 7 juxtaposed to FLI1 exon 6 (7.6); type 2, EWS exon 7 juxtaposed to FLI1 exon 5 (7.5); and type 3, EWS exon 10 juxtaposed to FLI1 exon 6 (10.6).

The high prevalence of EWS-ETS fusions in the Ewing's family of tumours and the large number of experimental evidences disclosed in the literature, suggest that the chimeric protein is a key factor in the tumour's genesis and maintenance.

Although in vitro experiments have shown the transforming ability of chimeric transcript expression, thus far the cell type wherein the translocation originates is not known. This lack of knowledge makes it difficult to understand tumour biology and makes it impossible to establish a progression pattern wherein molecular targets may be determined, without which it is difficult to develop an effective therapy. Therefore, establishing models aimed at understanding the molecular mechanisms of tumour transformation and determining the cell type wherein Ewing's tumour originates represent two of the most decisive objectives in research on this tumour.

The induction of the chimeric transcript in various cellular contexts has been one of the most widely used strategies aimed at elucidating the associated molecular differentiation processes, as well as the target genes which are directly induced by the fusion. Thus, studies have been conducted on various mouse cell lines:

NIH3T3

The NIH3T3 murine fibroblast cell line recapitulates fibroblast characteristics and represents a non-transformed line immortalised with the 3T3 protocol. Precisely because of the line's ease of growth and availability, it has been widely used as a cellular system for the induction of chimeric fusion. Amongst the most relevant conclusions arising from these studies, following can be highlighted: chimeric fusion promotes cell growth regardless of the anchor (May, W. A., et al., EWS/FLI1-Induced Manic Fringe Renders NIH-3T3 Cells Tumorigenic. Nat. Genet., 1997. 17(4): p. 495-7) and the formation of tumours in immuno-depressed mice (Thompson, A. D., et al., Divergent Ewing's Sarcoma EWS/ETS Fusions Confer a Common Tumorigenic Phenotype on NIH3T3 Cells. Oncogene, 1999. 18(40): p. 5506-13). That is, the induction of the chimeric transcript in immortalised cells confers tumorigenic capacity and deeply affects cellular morphology (Teitell, M. A. et al., EWS/ETS Fusion Genes Induce Epithelial and Neuroectodermal Differentiation. Lab Invest, 1999. 79(12): p. 1535-43).

C2C12

C2C12 (ATCC/CRL-1772) is a murine line of undifferentiated mesenchymal cells capable of differentiating in vitro into myoblasts and, by means of the adequate stimuli, into osteoblasts or adipocytes, characteristic phenotypes of a mesenchymal cell origin.

Chimeric fusion has been introduced in this cellular context in order to discover new genes that may be relevant in the differentiation processes associated with chimeric fusion and to recapitulate tumour histogenesis (Eliazer, S. et al., Alteration of Mesodermal Cell Differentiation by EWS/FLI1, the Oncogene Implicated in Ewing's Sarcoma. Mol. Cell. Biol. 2003 January; 23(2): 482-92).

In this cellular microenvironment, chimeric fusion is capable of drastically inhibiting the cell line's myogenic differentiation processes. The cells transfected with the fusion showed a significant delay in the expression of MyoD and a very low expression or a total absence of MyoD-induced genes, such as myogenin, desmin and actin. However, although the transfected cells also did not follow an osteogenic differentiation pattern, they were positive for alkaline phosphatase despite the fact that they did not show a constitutive increase in other bone markers, such as osteocalcin or type I collagen. Consequently, there is a disturbance of the process of differentiation into osteoblasts and myocytes in the C2C12 line.

Cellular morphology was subtly altered, since the transfected cells acquired a more cubic morphology that was reminiscent of human tumours and was very different from the muscle's typical fibrillar morphology.

The induction of fusion also modified the expression of cell cycle regulation genes. Two important molecules for passage through the G1 cell cycle restriction point were affected. P₂₁^{WAFI/CIP1} drastically reduced its expression, whilst the expression of cyclin D1 was significantly reinforced.

Primary Mouse Cells

Curiously, chimeric fusion is not capable of transforming mouse fibroblast primary cultures and immortalised fibroblast lines without co-operating mutations (Turc, et al., Chromosome Study of Ewing's Sarcoma (ES) Cell Lines. Consistency of a Reciprocal Translocation t(11; 22) (q24; q12). Cancer Genet. Cytogent., 1984. 12(1): p. 1-19).

Normal mouse cells into which chimeric fusion has been introduced are not capable of sustaining stable expression of the chimeric protein, probably due to the latter's toxic effect in this non-permissive cellular environment. This phenomenon has also been observed in immortalised cell lines from mesenchymatous (Rat-1), epithelial (HeLa) and neuroectodermal (NCM-1) cell lineages. Furthermore, the introduction of chimeric fusion in normal mouse fibroblasts surprisingly leads to the induction of apoptosis and cell cycle failure. This phenomenon is common to other oncogenes, such as c-Myc or Ras; cell apoptosis and growth inhibition have also been observed after they have been introduced into normal fibroblasts (Delattre, O. et al., Gene Fusion with an ETS DNA-Binding Domain Caused by Chromosome Translocation in Human Tumours. Nature, 1992. 359(6391): p. 162-5; Plougastel, B., et al., Genomic Structure of the EWS Gene and Its Relationship to EWSR1, a Site of Tumour-Associated Chromosome Translocation. Genomics, 1993, 18(3): p. 609-15). However, the interruption of the p19^{ARF} and p53 signalling pathways or the expression of genes E6 and E7 of the human papilloma virus allow for stable expression of the protein in normal fibroblasts. Furthermore, these secondary alterations reduce chimeric-protein-induced apoptosis and promote EWS-FLI1-induced tumorigenesis in normal mouse fibroblasts.

Multipotent Cells Derived from Human Bone Marrow Stroma (MCS)

Fusion transfection in multipotent human cells has also been used as a model to recapitulate tumour histogenesis.

MCS cells are capable of differentiating into multiple cell types, such as osteoblasts, adipocytes, chondrocytes, skeletal muscle fibres, flat muscle, neurons and astrocytes. In this model, the induction of chimeric fusion blocks osteoblastic and adipocyte differentiation. It is speculated that blockage of the differentiation processes is a tumorigenic mechanism directed by the chimeric protein that is relevant in tumour pathogeny (Plougastel, B., et al., Cloning and Chromosome Localization of the Mouse EWS Gene. Genomics, 1994, 23(1): p. 278-81). Furthermore, as is the case in other models, the transfected cells exhibited a more rounded morphology and the nucleus occupied a central position.

By means of specific mutations in one of the chimeric protein regions, the authors demonstrated that the presence of the ETS domain of FLI-1 is determinant for inhibition of osteoblastic differentiation, whilst it is not essential for blockage of differentiation into fatty tissue.

### Animal Models

On the other hand, the development of animal models makes it possible to recapitulate many features of tumour growth and development, such as the acquisition of genetic alterations associated with progression phenomena, or a tumour's capacity to generate metastasis. However, the formation of new tumours from cell lines or human tumours is complicated due to the cells' inability to grow in the new cellular microenvironment.

Animal models for Ewing's tumour are scarcer than animal models for other paediatric solid tumours, such as neuroblastoma or rhabdomyosarcoma (Beltlinger, C. et al., Murine Models for Experimental Therapy of Pediatric Solid Tumours with Poor Prognosis. Int. J. Cancer, 2001. 92(3): p. 313-8).

It is known that the capacity to form tumours is completely dependent on the immune system's activity. The development of tumours in normal mice has only been possible after treating them with immuno-suppressant agents. This treatment, unlike that of other tumours, such as osteosarcomas, does not modify the derived tumours' histology (Floersheim, G. L. et al., Growth of Human Tumours in Mice after Short-Term Immuno-suppression with Procarbazine, Cyclophosphamide, and Antilymphocyte Serum. Transplantation, 1980. 30(4): p. 275-80). It is even known that tumour formation is more efficient in athymic mice after inhibition of their macrophages and/or natural killer cells. In fact, tumour formation increases by over 60% after blockage of these cell types (Torhorst, J., Growth Stimulation in a Ewing Sarcoma after “Macrophage Blockade” in Athymic (Nude) Mice. Schweiz Med. Wochenschr., 1981. 111(36): p. 1319-21).

Amongst the models, one may highlight xenotransplants by administration of human Ewing cells to immuno-depressed mice. The lack of knowledge about the cell type wherein Ewing's tumour originates is perhaps the greatest obstacle to the development of new models.

The lack of good syngeneic models makes it impossible to develop potentially useful therapeutic strategies.

### DESCRIPTION OF THE INVENTION

One method to generate tumour models in animals consists of directly administering cells that express the genetic alterations characteristic of the tumour to the animal's body part in which the tumour in question forms. This makes it possible to study tumour development in situ.

In order to do so, it is possible to use cell lines or stem cells that have previously been transformed with the characteristic marker.

This invention relates to a method designed to obtain a non-human animal model for sarcoma characterised in that it comprises:
transfecting multipotent cells of mesenchymal origin, different from human embryonic cell lines, with an expression vector that contains a polynucleotide which encodes a fusion protein selected from EWS-FLI1, EWS-ERG, EWS-ETV1, EWS-ETV4, EWS-FEV, EWS-ATF1, EWS-WT1, EWS-NR4A3 and EWS-DDIT3; and
injecting said transfected cells in the animal that is the model subject.

In order to obtain the sarcoma model to which the invention relates, it is possible to use any type of multipotent mesenchymal cell line. As used in this invention, the terms “multipotent mesenchymal cell line” and “multipotent cell of mesenchymal origin” relate to any cell which, through the induction of a cellular differentiation genetic programme under the adequate stimuli, is capable of generating various mesenchymal cell types (within the same embryonic layer), such as muscle cells or myoblasts, adipocytes, chondrocytes' and/or osteoblasts. Said terms also include bone-marrow-derived multipotent adult progenitor cells (MAPC), which may generate or give rise to different cell types, both mesenchymal and other cell types pertaining to other embryonic layers different from their own.

In a preferred embodiment of the invention, said multipotent mesenchymal cell line is the C3H/10T1/2 cell line (ATCC/CCL-226).

According to the invention, the multipotent cell line is transfected with an expression vector that encodes a fusion protein, which comprises a fragment of the EWS protein. In a particular embodiment, said fusion protein has been selected from EWS-FLI1, EWS-ERG, EWS-ETV1, EWS-ETV4, EWS-FEV, EWS-ATF1, EWS-WT1, EWS-NR4A3 and EWS-DDIT3.

The sarcoma in the method of the invention may be any sarcoma whose characteristic chromosomal alteration leads to the expression of a fusion protein which comprises part of the EWS gene. In a particular embodiment, said sarcoma is a Ewing's tumour, clear cell sarcoma, small round cell desmoplastic tumour, myxoid chondrosarcoma or myxoid liposarcoma. The selection of the type of fusion protein is performed on the basis of the sarcoma model that is to be developed. In a particular embodiment, the selection is performed according to the following table:

TABLE 1
Types of sarcomas and their associated alterations

| | Characteristic alteration | | |
|---|---|---|---|
| Type of sarcoma | Fusion protein | Translocation | |
| Ewing's tumour | EWS-FLI1 | t(11: 22) | (q24; q12) |
| | EWS-ERG | t(21; 22) | (q22; q12) |
| | EWS-ETV1 | t(7; 22) | (p22; q12) |
| | EWS-ETV4 | t(17; 22) | (q21; q12) |
| | EWS-FEV | t(2; 22) | (q33; q12) |
| Clear cell sarcoma | EWS-ATF1 | t(12; 22) | (q13; q12) |
| Small round cell | EWS-WT1 | t(11; 22) | (p13: q12) |
| desmoplastic tumour | | | |
| Myxoid chondrosarcoma | EWS-NR4A3 | t(9; 22) | (q22-31; q11-12) |
| Myxoid liposarcoma | EWS-DDIT3 | t(12; 22) | (q13; q12) |

In a particular embodiment, the method of the invention is a method designed to obtain a non-human animal model for Ewing's tumour characterised in that it comprises:
transfecting multipotent cells of mesenchymal origin, different from human embryonic cell lines, with an expression vector that contains a polynucleotide which encodes a fusion protein selected from EWS-FLI1 type 1, EWS-FLI1 type 2, and EWS-FLI1 type 3; and
injecting said transfected cells in the animal that is the model subject.

In a particular embodiment, the method disclosed in this application is characterised in that the animal in the model is a rodent. In a preferred embodiment of this invention, said animal is an immuno-depressed animal. In a preferred embodiment, said animal is an immuno-depressed rodent.

In a more preferred embodiment of the invention, said method is characterised in that the animal is an athymic nude mouse.

In a preferred embodiment of the invention, said method is characterised in that said multipotent cells of mesenchymal origin are the C3H/10T1/2 cell line (ATCC/CCL-226). In this case, the C3H/10T1/2 line of mesenchymal origin is capable of differentiating, after induction with the chimeric transcript, into other aberrant cell types that are reminiscent of a neuroectodermal origin, due to the presence of specific neuronal markers such as Neurofilament M, Neu and tubulin III.

In another particular embodiment of the invention, the transfected cells are injected by intramuscular route.

In a particular embodiment of the invention, the method designed to obtain an animal model for sarcoma, for example, Ewing's tumour, is characterised in that the polynucleotide which expresses the fusion protein is operatively linked to an inducible transactivator, whose expression regulates the transcription of the polynucleotide that encodes said fusion protein. In a preferred embodiment of the method described herein, said inducible transactivator is an rtTA transactivator, regulated by tetracycline or by other products pertaining to the tetracycline family, such as doxycycline.

Besides, another subject of this invention is a non-human animal model for sarcoma, characterised in that it is obtained by means of:
the transfection of multipotent cells of mesenchymal origin, different from human embryonic cell lines, with an expression vector that contains a polynucleotide which encodes a fusion protein selected from EWS-FLI1, EWS-ERG, EWS-ETV1, EWS-ETV4, EWS-FEV, EWS-ATF1, EWS-WT1, EWS-NR4A3 and EWS-DDIT3; and
the injection of said transfected cells in the animal that is the model subject.

The sarcoma model of the invention may be any sarcoma whose characteristic chromosomal alteration leads to the expression of a fusion protein which comprises part of the EWS gene. In a particular embodiment, said sarcoma is a Ewing's tumour, clear cell sarcoma, small round cell desmoplastic tumour, myxoid chondrosarcoma or myxoid liposarcoma.

In a particular embodiment, the non-human animal model is a model for Ewing's tumour, characterised in that it is obtained by means of:
the transfection of multipotent cells of mesenchymal origin, different from human embryonic cell lines, with an expression vector that contains a polynucleotide which encodes a fusion protein selected from EWS-FLI1 type 1, EWS-FLI1 type 2, and EWS-FLI1 type 3; and
the injection of said transfected cells in the animal that is the model subject.

In a particular embodiment, said animal model is a rodent. In a preferred embodiment, said model is an immuno-depressed animal. In a more preferred embodiment, said animal model is an athymic nude mouse.

In a particular embodiment of this model, said multipotent cells of mesenchymal origin are the C3H/10T1/2 cell line (ATCC/CCL-226).

In a preferred embodiment of this animal model, the polynucleotide that expresses the fusion protein is operatively linked to an inducible transactivator, for example, rtTA, whose expression regulates the transcription of the polynucleotide that encodes said fusion protein.

Furthermore, this invention relates to the use of a non-human animal model for sarcoma, preferably Ewing's tumour, obtained by the method described above, in order to test a compound's pharmacological anti-tumour activity.

In a particular embodiment, said invention relates to the use of a non-human animal model of sarcoma, preferably Ewing's tumour, in a method to select compounds with anti-tumour activity characterised in that it comprises:
administering the compound whose anti-tumour activity is to be tested, to said model animal,
comparing the effect of said compound on said animal's tumours to the tumours of control animals that have not been treated with said compound.

Similarly, a preferred embodiment of the invention relates to the use of a non-human animal model for sarcoma, preferably Ewing's tumour, previously described, in a method designed to identify markers associated with said sarcoma, characterised in that it comprises comparing the presence, or absence, or the level of gene expression in tumour tissue samples of said model animal to the gene expression in the tissue of a second animal that has not been injected with transfected cells.

The invention also relates to a transformed cell line characterised in that it is a cell line derived from multipotent cells of mesenchymal origin different from human embryonic cell lines, transfected with an expression vector that contains a polynucleotide which encodes a fusion protein selected from EWS-FLI1, EWS-ERG, EWS-ETV1, EWS-ETV4, EWS-FEV, EWS-ATF1, EWS-WT1, EWS-NR4A3 and EWS-DDIT3.

In a preferred embodiment, the expression vector polynucleotide encodes a fusion protein selected from EWS-FLI1 type 1, EWS-FLI1 type 2, and EWS-FLI1 type 3.

In a particular embodiment, said multipotent cells of mesenchymal origin are a line of adult progenitor stem cells (MAPC).

In another particular embodiment of the invention, said transformed cell line originates from the C3H/10T1/2 cell line (ATCC/CCL-226).

Similarly, in a preferred embodiment of the invention, said cell line, previously described, is characterised in that the polynucleotide which expresses the fusion protein is operatively linked to an inducible transactivator, whose expression regulates the transcription of the polynucleotide that encodes said fusion protein. In a preferred embodiment, said cell line is characterised in that said inducible transactivator is rtTA.

Finally, the present invention also relates to the use of the previously described transformed cell lines, in an in vitro method designed to test a compound's anti-tumour activity, characterised in that it comprises:
culturing a sample of said cells with the compound to be tested; and
evaluating the effect of said compound on said cells by analysing genotypic or phenotypic features of interest with respect to a sample of the same cells that has not been treated with said compound.

### BRIEF DESCRIPTION OF THE FIGURES

FIG. 1. Activation mechanisms for the retroviral vector. In the presence of Doxycycline (Dox+), the transactivating protein (rtTA) is capable of recognising the tetO region, which allows for transcription of the gene of interest under the Cytomegalovirus promoter.

FIG. 2. Analysis of the selected clones. A. The upper figure shows the absence of expression of chimeric fusion in both parental cells and those transfected with the empty vector. The lower gel shows the PCR product load for Beta-actin, which was used to normalise the expression levels. The positive control used pertained to a Ewing's Tumour cell line with a EWS-FLI1 type 2 fusion. B. The figure shows the expression of chimeric fusion in different clones. Clones 7.5, 10.6 and 10.6 ET were positive for chimeric fusion following induction of the vector with doxycycline (2 μg/ml of Dox, 48 h). All the clones exhibited regulated expression, with the expression of fusion being null in the absence of doxycycline. Clone 10.6 ET showed higher fusion expression levels than clones 7.5 and 10.6. In addition, other clones carrying type 7.6 and 7.5 inserts were isolated which did not exhibit regulated expression of fusion and were, therefore, discarded from the study. The amplification of Beta-actin was used to normalise the fusion expression levels.

FIG. 3. Induction of chimeric fusion by Doxycycline. A. Upper gel. Induction of chimeric fusion following treatment of the cells with increasing doxycycline doses (0-3 μg/ml) in clone 7.5. The maximum induction was obtained for 2.5-μg/ml doxycycline doses. Lower gel. The quantity of amplified product was identical in all cases, as shown by Beta-actin. B. Upper gel. The maximum induction for clone 10.6 was at 1.5-μg/ml doxycycline doses. Higher doses caused saturation of the promoter's response. Lower gel. Beta-actin.

FIG. 4. Immuno-precipitation of clones 7.5 and 10.6. The EWS-FLI1 protein was expressed in a regulated manner in clones 7.5 and 10.6. In both cases the samples that were not treated with doxycycline and those with the clone carrying the empty vector, the antibody detected a slight band with the same molecular weight. The A673 cell line was included as a positive control in the experiment.

### EMBODIMENT OF THE INVENTION

The present invention is additionally illustrated with the following example, which represents a particular embodiment thereof, intended to illustrate it and in no way limited thereto.

### Example 1

The murine C3H/10T1/2 cell line was obtained from ATCC (ATCC/CCL-226). This line was used as the parental cell line in the cellular transformation studies. It was isolated in 1973, from mouse embryonic cells (C3H strain). It exhibits a fibroblastic morphology, grows in adherence, and is sensitive to contact inhibition. It is a pluripotent murine cell line, which may differentiate, by means of specific inducers, into osteoblasts, chondrocytes, adipocytes and myoblasts.

### 1.—Construction of the Inducible Vector.

In order to generate the inducible model, a retroviral vector regulated by tetracycline or any of its analogous compounds (doxycycline) was used.

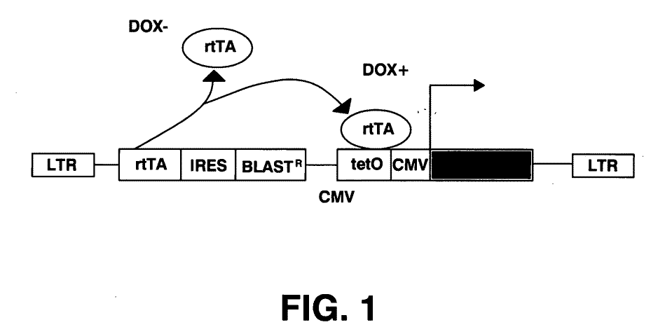

This vector consists of two complementary expression systems. One of the systems is an expression vector for the transactivating protein (rtTA), which constituent expresses this protein. The other system consists of a doxycycline-inducible expression system which, when active, leads to the transcription of the gene of interest, under the cytomegalovirus (CMV) promoter. Furthermore, the vector exhibits two ampicillin and blasticidin resistance genes for its selection in prokaryote land eukaryote cells, respectively. In the presence of the effector (doxycycline), the rtTA protein is capable of recognising its target sequence (tetO) within the vector, thus allowing for the transcriptional activation of the gene of interest (FIG. 1), originally developed by Watsuji and collaborators (Watsuji, T. et al., Controlled Gene Expression with a Reverse Tetracycline-Regulated Retroviral Vector (RTRV) System. Biochem. Biophys. Res. Commun., 1997. 234(3): p. 769-73).

### a) Amplification

At first, multiple copies of the inserts (EWS-FLI-1 fusions; types 1, 2 and 3) originally cloned in the pcDNA 3.1+/− vector were obtained by means of PCR (Lin P P, et al. Differential Transactivation by Alternative EWS-FLI1 Fusion Proteins Correlates with Clinical Heterogeneity in Ewing's Sarcoma; Cancer Res. 1999; 54: 1428-32).

To achieve this, a high-fidelity DNA polymerase enzyme was used (Invitrogen/11304-011). The primers used for the amplification were designed to endow the inserts with two restriction targets useful for their subsequent cloning in the retroviral vector. The amplicon thus obtained introduced a restriction target for the Xho I enzyme in sequence 5′ (CTCGAG) and a restriction target for Not I on the 3′ end (CGGCCGC). Table 1 shows its sequence and its hybridization temperature (HT).

TABLE 2
Sequence and hybridization temperature of the
primers used for the amplification of the
chimeric transcript.

| | SEQUENCE | |
|---|---|---|
| PRIMER | 5′ . . . 3′ | HT |
| EWS (S) | AAAAAAACTCGAGGAGAACGAGGAGGAAGGAGAG | 58° C. |
| | ▴ | |
| FLI (AS) | AAAAAAAAAAGCGGCCGCTCAGTAGTAGCTGCC | |
| | ▴ | |

S: sense; AS: anti-sense.

The amplification conditions were:

| | |
|---|---|
| Initial denaturation | 94° C. 5 min |
| Cycle reaction | 94° C. 30 s |
| (40 cycles) | 58° C. 45 s |
| | 72° C. 3 min |
| Final extension | 72° C. 7 min |

Once the gel was developed and it was verified that inserts had the correct size, the bands pertaining to the amplification products were extracted from the gel and purified using the Qiagen commercial method, Qiagen gel extraction kit (Qiagen/28704), following the manufacturer's instructions.

### b) Digestion

Once purified, the products were digested with restriction enzymes Xho I and Not I. The original retroviral vector, which carried the targets for the enzymes, was also digested.

The digestion reaction consisted of a mixture, whose final composition was:
Retroviral vector (0.5 μg) or Insert (3 μl of PCR product for each type of fusion)
Digestion buffer (50 mM Tris-HCl, 1 mM MgCl₂, 100 mM NaCl, 1 mM DTT, pH 7.9)
1% BSA
Xho 1 (0.5 μl/New England Biolabs/R0146S)
Not 1 (0.5 μl/New England Biolabs/R0189S)
Water to complete a 30-μl volume

The digestion took place at 37° C. for 20 hours.

Each of the digested products was re-run on a 1% agarose gel and, once its size was verified, was extracted from the gel and purified as previously described.

### c) Ligation

The ligation reaction on the purified products took place at 15° C. overnight, using 1 μl of Ligase T4 (New England Biolabs/202) and 1 μl of 10× ligation buffer supplied by said company. For the reaction, 0.5 μl of previously digested retroviral vector were used, and between 0 and 1 μl for each type of insert. As a control, the same reaction was performed without including the insert.

### d) Transformation, Selection and Obtaining of the Retroviral Vector

All the ligation product for each of the samples was used to transform the E. coli Top®10 bacteria (Invitrogen/K4575-01), following the manufacturer's instructions. Once transformed, they were seeded in agar plates with ampicillin (50 μg/ml). Several ampicillin-resistant clones were selected for each type of insert and grown separately in 5 ml of liquid LB (DIFCO/214050) overnight with shaking (300 rpm) at 37° C.

An analysis of 4/5 colonies was conducted for each of the 3 ligations, using the Roche commercial kit. After obtaining the plasmid DNA, an aliquot of each of the plasmids was digested with Xho I and Not I as previously described. The digestion product was run on an agarose gel and the colonies that tested positive for the presence of inserts were detected. Subsequently, sequencing by fluorescence was conducted.

Once the DNA sequence fidelity and integrity was verified for each of the fusions following the ligation reaction, the plasmids were grown in a greater volume of LB broth (200 ml), containing ampicillin at a concentration of 100 μg per ml, which was allowed to grow for 16 hours at 37° C. and 300 rpm.

The plasmid DNAs were extracted using the Clontech method (Clontech/PT3178-2). They were re-suspended at a concentration of 1 μg/ml in TE buffer and frozen at −20° C. until their subsequent use.

### 2.—Obtaining of Transfectant Retroviruses

In order to obtain viral particles, the AmphoPack™ cell line (BD Biosciences C3201-1) was transfected.

The maximum transfection efficiency conditions were determined by means of a previous experiment in which the same cell line was transfected, with a vector carrying the β-galactosidase gene.

In order to perform the transfections, LIPOFECTAMINE™ 2000 (Invitrogen/11668-027) was used. The transfection was performed in cells grown in 6-well plates (CORNING/3506) to an 80% confluence. To this end, the cells were left in 2 ml of OPTI-MEM® medium with Glutamax (Invitrogen/51985-026) per well. For each type of fusion, 8 μg of DNA of the vector with insert diluted in a 500-11 volume of OPTI-MEM medium were mixed with 20 μl of LIPOFECTAMINE™ 2000, also diluted in 500 μl of OPTI-MEM, following the manufacturer's instructions. As controls, the transfection of the empty vector (without insert), and with the LIPOFECTAMINE™ 2000 solution without any type of plasmid DNA were performed.

The two solutions (medium; vector and medium; lipofectamine) were mixed and incubated for 30 min at room temperature (RT). The mixture was deposited on the cells. The transfection was performed in this OPTI-MEM medium without fetal serum (FS) overnight, after which time the transfection medium was replaced by complete medium. After 48 hours, the medium was collected, centrifuged, filtered using a filter with a pore size of 45 microns and stored at −70° C. until it was used.

### 3.—Transduction

The C3H/10T1/2 cell line was cultured in 6-well plates, at a density of 1×10⁵ cells/well. After 24 hours, the supernatant obtained from the transfection of the AmphoPack™ cells, previously centrifuged and diluted 1:2, 1:4 and 1:6 in complete culture medium, was added. After 24 hours in the presence of this diluted supernatant, the medium was replaced by complete medium.

### 4.—Selection of Clones

After 48 hours, the selection process began using treatment with blasticidin (2 μg/ml), in order to select those cells that were positively infected with the cell virions.

The selective blasticidin dose was established on the basis of a previous cytotoxicity experiment for the C3H/10T1/2 parental cell line, which received increasing doses of blasticidin (0.3, 0.6, 0.8, 1, 1.5, 2, 2.5, 3 μg/ml). 2 μg/ml was determined to be the lowest dose that induced 100% death due to cytotoxicity.

Approximately two weeks after transfection, those clones containing over 200 cells were isolated. Between 5-8 clones were collected for each type of fusion by means of filter disks (Pgc Scientifics/17-2). To this end, the plates were washed with PBS and the filter disks, slightly impregnated with trypsin-EDTA, were placed over the clones. After 5-10 minutes in the incubator at 37° C., they were transferred, using forceps, to a 24-well plate containing complete medium. It was softly stirred in order to facilitate cell distribution in the well. All the clones were grown until a sufficient number of cells were obtained for freezing. Prior to the molecular analysis of the transfectants, these were grown for at least one-and-a-half months in the presence of an antibiotic. Furthermore, cell passages during this selection time were conducted at low density.

### Analysis of the Selected Clones.

The presence of chimeric fusion in the clones obtained from selection with blasticidin was analysed by means of RT-PCR.

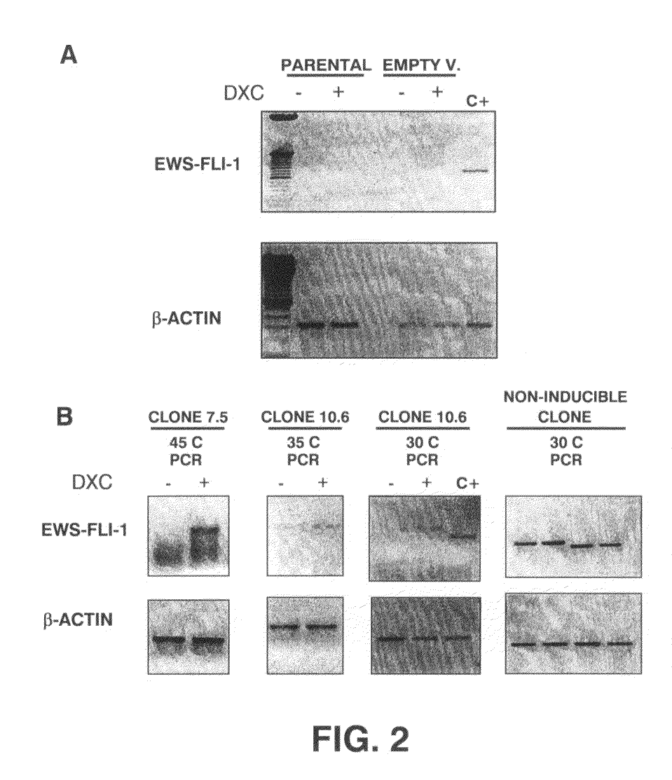

Non-transduced parental cells and cells transduced with the empty vector that tested negative for the EWS-FLI1 fusion were used as controls. Using the amplification primers, positive clones were obtained for the three types of fusions (7.5, 7.6 and 10.6). However, inducible clones were only found for the type 2 (7.5) and type 3 (10.6) fusions (FIG. 2).

### Trans-Differentiation of Clone 10.6.

When the selected cells reached passage 36, antibiotic was once again added in order to verify culture clone-ability. The cells maintained their viability after addition of the antibiotic, thus showing that they continued to carry the retroviral vector. However, a spontaneous morphological change was observed in clone 10.6.

Within the cell culture, some cells phenotypically different from clone 10.6 appeared, which were designated as Ewing-Type (10.6 ET), due to their morphological similarity to the tumour cells. These cells were separated from the culture and were separately grown in the presence or absence of blasticidin, in order to determine whether their phenotype varied. The isolated cells maintained their phenotype constant through time, regardless of the presence or absence of blasticidin in the culture medium. Molecular analysis of this subclone revealed the presence of a EWS-FLI1 type 3 fusion, identical to clone 10.6, from which it was derived. However, surprisingly, the RT-PCR studies showed that the expression levels of chimeric fusion were greater in this subclone than in the 10.6 clone from which it derived, since a lower number of PCR cycles was necessary to achieve a similar amplification level.

### Induction of Chimeric Fusion by Doxycycline.

Clones 7.5 and 10.6 received increasing doses of doxycycline for 48 hours, after which time the presence of fusion was examined by means of RT-PCR.

For clone 7.5, 0.3 μg/ml-doxycycline doses were capable of causing a slight induction that was dose-dependent, with it reaching a maximum at doxycycline concentrations of 2.5 μg/ml.

In clone 10.6, induction of fusion was also found at doses greater than 0.3 μg/ml of doxycycline. Concentrations greater than 1.5-2 μg/ml were unable to cause greater induction of the chimeric transcript.

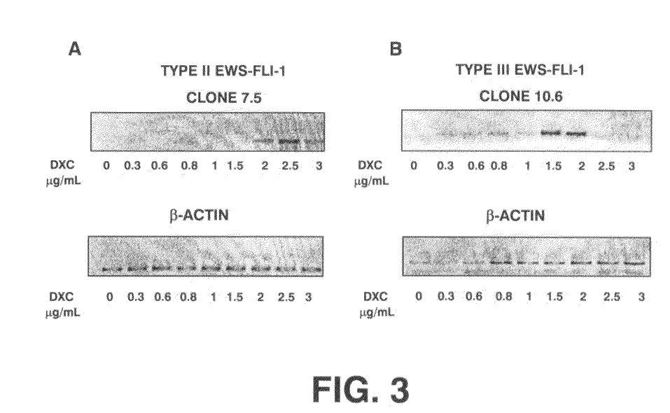

In the absence of doxycycline, the vector was inhibited and both clones tested negative for chimeric fusion (FIG. 3).

### Immuno-Precipitation.

The presence of the EWS-FLI1 protein in clones 7.5 and 10.6 was analysed by means of immuno-precipitation. Furthermore, the clone carrying the empty vector and the A673 Ewing tumour cell line were included as controls in the experiment.

Cell lysis of clones 7.5 and 10.6 (treated with or without doxycycline, DXC+/−, respectively) were immuno-precipitated with an anti-FLI1 antibody. Following development of the gel and transfer of the immuno-precipitated protein, detection was performed with an anti-EWS antibody.

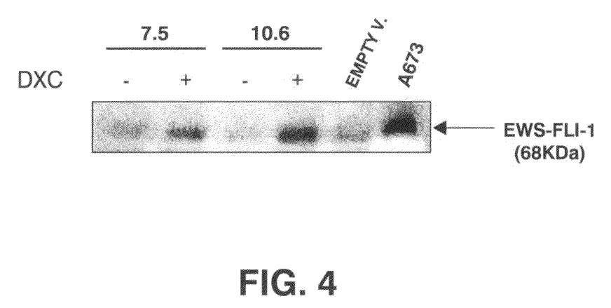

The detection disclosed that both clones expressed the chimeric protein in a regulated manner, since the expression levels were much higher in the presence of doxycycline. A slight band with the same molecular weight as the chimeric protein was observed, both in the samples of clones that were not stimulated with doxycycline and in the cells transfected with the empty vector (FIG. 4).

Since it may be regulated, this expression system has the following advantages:

1.—It makes it possible to discriminate whether it is necessary for there to be a transcript “expression threshold” in order for the transcript to relate to a given phenotype.
2.—It makes it possible to know whether there are early/late fusion-regulated genes that may be involved in tumour initiation/progression mechanisms.

In the above-mentioned studies conducted on the C2C12 cell line and the mouse pluripotent mesenchymal line, stable expression systems are used, and in neither of the two cases is transformation of the transfected cells demonstrated either in vivo or in vitro.

### Example 2

In the animal model developed, a total of 96 athymic nude mice were used, divided in the following groups:

For each of the clones under study (EWS-FLI-1 types 1, 2 and 3 fusions), 24 mice were used for each type of fusion. These groups were subdivided into 2 subgroups with 12 animals/group, depending on whether or not they were given doxycycline. The administered doxycycline dose was 1.5 mg/ml in the drinking water.

For those mice that were injected with parental cells or cells transformed with the empty vector, 16 mice per cell type (parental/empty vector) were used, which were again subdivided into two subgroups with 8 mice/group, depending on whether or not they were given doxycycline in the drinking water.

Each mouse received an intramuscular injection in the posterior tibial gastronemius muscle, using a 29G needle, of 200,000 cells obtained from an exponential cell culture re-suspended in 100 μl of sterile PBS, which exhibited a viability rate greater than 95%.

The results obtained were the following:

TABLE 3
Induction of tumours in vivo

| | EWS-FLI-1 | EWS-FLI-1 | EWS-FLI-1 |
|---|---|---|---|
| | TYPE 1 | TYPE 2 | TYPE 3 |
| | 7.5 | 7.6 | 10.6 |
| INCIDENCE | (22/24) −92% | (24/24) −100% | (12/24) −50% |
| LATENCY TIME | 15 ± 6.2 | 25 ± 5.6 | 53 ± 5.5 |
| (days) | | | |

None of the mice who had been injected with parental cells neither those transformed with the empty vector developed tumours.

As shown in the table, the three clones were capable of growing in vivo. The tumour efficiency was as high as 100% for type 2 fusions, which stresses the high tumorigenic efficiency of those cells transformed with the chimeric transcript.

1-29. (canceled)

30. A method designed to obtain a non-human animal model for sarcoma, comprising:
transfecting multipotent cells of mesenchymal origin, different from human embryonic cell lines, with an expression vector that contains a polynucleotide which encodes a fusion protein selected from EWS-FLI1, EWS-ERG, EWS-ETV1, EWS-ETV4, EWS-FEV, EWS-ATF1, EWS-WT1, EWS-NR4A3 and EWS-DDIT3; and
injecting said transfected cells in the animal to become the model subject.

31. A method, as claimed in claim 30, wherein the sarcoma is selected from: Ewing's tumour, clear cell sarcoma, small round cell desmoplastic tumour, myxoid chondrosarcoma or myxoid liposarcoma.

32. A method designed to obtain a non-human animal model for Ewing's tumour, comprising:
transfecting multipotent cells of mesenchymal origin, different from human embryonic cell lines, with an expression vector that contains a polynucleotide which encodes a fusion protein selected from EWS-FLI1 type 1, EWS-FLI1 type 2, and EWS-FLI1 type 3; and
injecting said transfected cells in the animal to become the model subject.

33. A method, as claimed in claim 30 or 32, wherein the animal subject of said model is a rodent.

34. A method, as claimed in claim 30 or 32, wherein the animal is an immuno-depressed animal.

35. A method, as claimed in claim 30 or 32, wherein said animal is an immuno-depressed mice.

36. A method, as claimed in claim 30 or 32, wherein the animal is an athymic nude mouse.

37. A method, as claimed in claim 30 or 32, wherein the multipotent mesenchymal cell line is the C3H/10T1/2 cell line (ATCC/CCL-226).

38. A method, as claimed in claim 30 or 32, wherein said transfected cells are injected intramuscularly.

39. A method, as claimed in claim 30 or 32, wherein the polynucleotide which expresses the fusion protein is operatively linked to an inducible transactivator, whose expression regulates the transcription of the polynucleotide that encodes said fusion protein.

40. A method, as claimed in claim 39, wherein said inducible transactivator is an rtTA transactivator.

41. A non-human animal model for sarcoma obtained by means of:
the transfection of multipotent cells of mesenchymal origin, different from human embryonic cell lines, with an expression vector that contains a polynucleotide which encodes a fusion protein selected from EWS-FLI1, EWS-ERG, EWS-ETV1, EWS-ETV4, EWS-FEV, EWS-ATF1, EWS-WT1, EWS-NR4A3 and EWS-DDIT3; and
the injection of said transfected cells in the animal to become the model subject.

42. A non-human animal model for Ewing's tumour obtained by means of:
the transfection of multipotent cells of mesenchymal origin, different from human embryonic cell lines, with an expression vector that contains a polynucleotide which encodes a fusion protein selected from EWS-FLI1 type 1, EWS-FLI1 type 2, and EWS-FLI1 type 3; and
the injection of said transfected cells in the animal to become the model subject.

43. An animal model, as claimed in claim 41 or 42, wherein said animal is a rodent.

44. An animal model, as claimed in claim 41 or 42, wherein said animal is an immuno-depressed animal.

45. An animal model, as claimed in claim 41 or 42, wherein said animal is an immuno-depressed mice.

46. An animal model, as claimed in claim 41 or 42, wherein said animal is an athymic nude mouse.

47. An animal model, as claimed in claim 41 or 42, wherein said multipotent cells of mesenchymal origin are the C3H/10T1/2 cell line (ATCC/CCL-226).

48. An animal model, as claimed in claim 41 or 42, wherein the polynucleotide which expresses the fusion protein is operatively linked to an inducible transactivator.

49. An animal model, as claimed in claim 48, wherein said inducible transactivator is rtTA.

50. A method to test a compound's pharmacological anti-tumor activity, which comprises administering the compound to the non-human animal model of sarcoma, defined in claim 41 or 42.

51. The method as claimed in claim 50 comprising:
administering the compound whose anti-tumour activity is to be tested, to said model animal, and
comparing the effect of said compound on said animal's tumour against else tumours of control animals that have not been treated with said compound.

52. A method designed to identify markers associated with a sarcoma, comprising comparing the presence, or absence, of the gene expression level in tumour tissue samples of the non-human animal model of sarcoma defined in claim 41 or 42 against the gene expression level in the tissue of a second animal that has not been injected with transfected cells.

53. The method as claimed in claim 50, 51 or 52, wherein the sarcoma is an Ewing's tumour.

54. A transformed cell line derived from multipotent cells of mesenchymal origin different from human embryonic cell lines, transfected with an expression vector that contains a polynucleotide which encodes a fusion protein selected from EWS-FLI1, EWS-ERG, EWS-ETV1, EWS-ETV4, EWS-FEV, EWS-ATF1, EWS-WT1, EWS-NR4A3 and EWS-DDIT3.

55. A cell line, as claimed in claim 54, transfected with an expression vector that contains a polynucleotide which encodes a fusion protein selected from EWS-FLI1 type 1, EWS-FLI1 type 2, and EWS-FLI1 type 3.

56. A cell line, as claimed in claim 54, wherein said cell line originates from the C3H/10T1/2 cell line (ATCC/CCL-226).

57. A cell line, as claimed in claim 54, wherein the polynucleotide which expresses the fusion protein is operatively linked to an inducible transactivator, whose expression regulates the transcription of the polynucleotide that encodes said fusion protein.

58. A cell line, as claimed in claim 57, wherein said inducible transactivator is rtTA.

59. An in vitro method designed to test a compound's anti-tumour activity, comprising:
culturing a sample of cells of the cell line defined in claim 54 with the compound to be tested; and
evaluating the effect of said compound on said cells by analysing genotypic or phenotypic features of interest with respect to a sample of the same cells that have not been treated with said compound.


SEQUENCE LISTING (2 sequences)

SEQ ID NO: 1 - DNA, 34 bp, Artificial Sequence
  Synthetic Construct; Synthetic Oligonucleotide
aaaaaaactcgaggagaacgaggaggaaggagag

SEQ ID NO: 2 - DNA, 33 bp, Artificial Sequence
  Synthetic Construct; Synthetic Oligonucleotide
aaaaaaaaaagcggccgctcagtagtagctgcc